(19) United States
(12) Patent Application Publication
(10) Pub. No.: US 2009/0023576 A1
(43) Pub. Date: Jan. 22, 2009

(54) Method for the surface treatment of bead polymers

(75) Inventor: Ottmar Kryts, Bad-Nauheim (DE)

Correspondence Address: DeMont & Breyer, LLC, 100 Commons Way, Ste. 250, Holmdel, NJ, 07733, US

(73) Assignee: BAUER TECHNOLOGIES GMBH, 56203 Hoehr-Grenzhausen (DE)

(21) Appl. No.: 11/911,307

(22) PCT Filed: Apr. 12, 2006
(86) PCT No.: PCT/EP06/03372

§ 371 Date: Apr. 21, 2008

(30) Foreign Application Priority Data
Apr. 16, 2005 (DE) 10 2005 017 586.4

(51) Int. Cl. A61K 6/087; A61K 6/08; C08F 120/18; B24B 1/00; C04B 38/00

(52) U.S. Cl. 501/80; 523/116; 526/329.7; 241/30; 241/23; 451/28

(57) ABSTRACT

A method for the surface treatment of bead polymers is to be prepared, by means of which bead polymers are treated in a particularly simple and effective manner such that upon addition to unfinished ceramic pieces, in particular, their strength and dimensional stability increase. To this end, the bead polymers are conducted to a device which is used for comminution, in particular, to a mill, in which the surface of the bead polymers is purposefully scratched or roughened in a uniformly mechanical manner.

The invention concerns a method for the surface treatment of bead polymers and the use of such surface-treated bead polymers.

Bead polymers are used in different branches, such as technology and medicine, as an additive for other plastics or ceramics, in order to produce new products or to optimize their manufacturing or product characteristics.

In the production of ceramic substrates up to now, bead polymers were added to the ceramic raw materials, and they were thermally removed in further processing. The temporarily added polymers were used to produce a definable porosity in the ceramic body.

The use of bead polymers was selected since they exhibit a particularly low liquid need in the starting ceramic composition, such as slurry, paste, or pulp. In this way, undesired shrinkage characteristics are avoided. Another advantage of the bead polymers is that they can be produced in exactly definable particle sizes.

In the processing of previously used bead polymers, however, it has been discovered that with increasing bead polymer content, the strength of the unfinished ceramic piece, the untempered component, rapidly declined so that there was no longer sufficient strength for further processing. This effect appeared, in particular, with fine structures, which even led to the appearance of layerings, and entire contact sites lost their inner firmness.

This effect is based on the tearing out of the bead polymers in places from the composite of the total matrix. In particular, this effect has manifested itself with components that are still moist. Also, the wear resistance of the thermally untreated unfinished piece has proved to be insufficient for many areas of application.

The use of so-called precipitation polymers or also of mechanically broken bead polymers leads to an increased liquid requirement, accompanied by a significant decline in plasticity, and consequently does not fulfill the desired requirements.

The goal of the invention therefore is to prepare a method by means of which bead polymers are treated, in a particularly simple and effective manner, so that they have a comparatively low liquid requirement and when added to unfinished ceramic pieces, in particular, do not cause a reduction in plasticity, but rather increase strength and dimensional stability.

In accordance with the invention, this goal is attained in that the bead polymers are conducted to a device which is used for comminution, in particular, a mill, in which the surface of the bead polymers is purposefully scratched or roughened in a uniform mechanical manner.

As a result of the bead polymers being processed by means of a device used for comminution in such a way that the comminution effect is suppressed to a large effect, the individual particles of the bead polymer exhibit uniformly distributed scratch traces with a predetermined depth on their surfaces after the processing. In comparison to chemical processing, for example, by etching, this has the advantage that chemical residues, which can influence the further course of the processing, do not remain behind.

With an increase in the scratches on the surfaces of the otherwise smooth bead polymers, the strength of the composition is increased without the plasticity of the composition being permanently disturbed. Also, the liquid requirement of the composition is not seriously increased. In particular, the particle size and the geometrical shaping of the polymers are retained without any significant change.

When dry, a reduced wear behavior of the scratched or roughened polymer surfaces is determined, and with the further thermal treatment of unfinished ceramic pieces, the starting composition receives a corresponding pore formation caused by the empty sites formed by the polymer in the ceramic matrix.

Surprisingly, it has been shown that devices used for the comminution from the category of mills, preferably, stone or disk, pin, splitting, impact or cross-beater mills, are normally particularly unsuitable for comminuting bead polymers, with a purposeful selection of the treatment parameters, such as usage time and operating speed of the individual processing tools, but rather they merely scratch or roughen them on the surface. In this way, especially the adhesion capacity of the individual bead polymer body is improved, for example, by interlocking or a greater friction of the bodies in or with the matrix. The consequences are an improved firmness and composite and an increased strength.

To improve undesired thermal deformation of the bead polymers and/or the undesired cementing of the bead polymers, for example, due to machine and/or friction heat during the surface treatment, the bead polymers or the device used for the comminution is/are preferably cooled. Added cooling or refrigerating agents, for example, which are subsequently removed once again, can also be used.

In a particularly advantageous development of the method, the bead polymers are cooled before they are supplied and/or while they are being supplied.

Such surface-treated bead polymers are preferably used as cavity-forming agents and/or as pore- or canal-forming agents in ceramic compositions.

By means of the addition of the scratched or roughened bead polymers in accordance with the invention, the endurance limit of the ceramics—also called “fatigue strength”—is improved.

As a result of the improved mechanical characteristics due to the use of such surface-treated bead polymers, their use plays a great role in medical technology, preferably, for the production of bone cements or dental cements.

In view of the need of as dense as possible a sphere packing in the arrangement of the polymers in the cement matrix, the use of bead polymers in this field is common. As bead polymer, polymethyl methacrylate (PMMA) is usually used. To this end, the polymer particles are mostly mixed, on site, with an X-ray contrast agent, a starter system, and possible active substances with the matrix liquid, the monomer of the polymer. During the setting of the cement, the polymer particles are bound into the solidifying matrix of the monomer, just like the other components.

By the addition of the scratched or roughened bead polymers in accordance with the invention, a substantially higher strength is produced in the cement composite than with untreated, smooth polymer parts. The endurance limit, in particular, is improved, in contrast to untreated polymer cements. This positive effect is manifested both in the so-called low-viscosity cements and in the so-called high-viscosity cements, just as in the so-called dental cements.

The normally feared fractures due to torn-out polymer beads from the cement matrix, so-called fatigue fractures, in the cements with traditional bead polymers are thus avoided by the use of such surface-treated scratched or roughened bead polymers. This is of special importance, particularly for a desired, long-lasting, or permanent stay of implants in the body.

In addition, the bead polymers, surface-treated according to the method of the invention, are preferably used as a filler in the lacquer and paint industries. These special lacquers and paints are particularly characterized by an improved wear resistance, as a result of the scratched or roughened bead polymers.

Another particularly advantageous use of such surface-treated bead polymers is to be found in their use as a surface coating, in particular, because of the anti-slip effect of such coatings.

The invention is explained in more detail, below, with the aid of the embodiment examples.

### EXAMPLE 1

A commercially available bead polymer based on methacrylate with an average particle size of 40 μm is cooled briefly and subsequently ground, in one or more passages, in a disk mill—similar to a flour mill—in which the disk slit is adjusted so as to be free of contact, wherein the grinding stone speed and the grinding stone interval are adjusted in such a way that a comminution of the material to be scratched, the bead polymer, does not occur or occurs only to a slight extent. The polymer treated in this way is added, as a filler and pore-forming agent, to the ceramic raw material, which is then treated further in a conventional manner.

### EXAMPLE 2

A commercially available polymethyl methacrylate of pharmaceutical quality is scratched, as a bead polymer, on the surface of the polymer beads in a pin mill. The rotational speed of the pins of the pin mill, the throughflow quantity of the bead polymer, and perhaps the quantity of air added as a transporting and cooling agent are adjusted in such a way that a comminution effect does not appear, or appears only insubstantially, and the polymer particles are scratched only superficially. The polymer thus obtained is blended with the other cement components—for bone cement or dental cement—and mixed with the monomer during usage and cured, wherein for dental use, so-called light-curing agents can also be used.

### EXAMPLE 3

A dyed bead polymer with embedded electrically conductive components is treated in an impact mill in such a way that the impact rate is below the particle destruction force and the beads of the polymer are scratched superficially on the impact surface. The impact surface is thereby cooled, in order to prevent a thermal deformation of the polymer. The polymer part thus scratched on its surface is admixed to a lacquer as a filler and processed conventionally to provide a wear-resistant, electrically conductive protective layer.

### EXAMPLE 4

A very low-viscous bead polymer is cooled in such a way that it is briefly frozen and, in this way, attains a sufficient hardness so as to scratch it on its polymer surface in a conical splitting mill. Subsequent to this, it is placed as an anti-slip cover in or on a surface to be coated and bound to it. The cover thus obtained can be kept extremely thin as a result of the surface characteristics of the polymer parts, since the adhesion with the substrate is very pronounced.

1. A method for the surface treatment of bead polymers, comprising:
conducting the bead polymers to a device which is used for comminution;
purposely scratching or roughening the surface of the bead polymers in a uniformly mechanical manner; and
cooling the bead polymers and/or the device which is used for comminution.

2. (canceled)

3. The method of claim 1, in which the bead polymers are cooled before they are supplied to the device used for comminution and/or while they are being supplied to the device.

4. A method for forming cavities and/or pores or canals in ceramic compositions, the method comprising utilizing, as agents, bead polymers which are surface-treated according to the method of claim 1.

5. A method for producing bone cements or dental cements, the method comprising utilizing bead polymers which are surface-treated according to the method of claim 1.

6. A method for providing a filler in the lacquer and dye industries, the method comprising utilizing bead polymers which are surface-treated according to the method of claim 1.

7. A method for providing a surface coating, the method comprising utilizing bead polymers which are surface-treated according to the method of claim 1.
